(19) United States
(12) Patent Application Publication
(10) Pub. No.: US 2026/0151764 A1
(43) Pub. Date: Jun. 4, 2026

(54) TEST KIT FOR HUMAN TISSUE

(71) Applicant: IN FUNG Co., Ltd., Taichung City (TW)

(72) Inventor: SHANG-YI WANG, Taichung City (TW)

(21) Appl. No.: 19/229,226

(22) Filed: Jun. 5, 2025

(30) Foreign Application Priority Data
Dec. 2, 2024 (TW) 113146612

(51) Int. Cl. B01L 3/00

(52) U.S. Cl. CPC B01L 3/5023; B01L 2200/025; B01L 2300/0825; B01L 2300/0887; B01L 2300/10; B01L 2300/126; B01L 2400/0481

(57) ABSTRACT

The present disclosure provides a seat, formed with a receiving groove on a top face thereof, being transparent at least at the receiving groove so that the receiving groove is visible from the outside; a test paper, disposed on a bottom of the receiving groove, being functional in testing and color indication so that a test result is able to be recognized by one outside; an adhesion layer, having an adhesion face for adhering onto the top face of the seat to cover the receiving groove; a pressing piece, having a first face and a second face, the first face being disposed on the adhesion face of the adhesion layer, the second face facing the seat and having plural protruding portions; after placing a human tissue into the receiving groove and making the adhesion layer adhere onto the top face of the seat, the second face of the pressing piece presses the human tissue to squeeze fluid in the human tissue to make the test paper wet so as to undergo a specific test and to be recognizable from a bottom of the seat.

### FIELD OF THE INVENTION

The present disclosure relates to a test kit to examine whether the human tissue has specific chemical or biological characteristics.

### BACKGROUND OF THE INVENTION

Stomach ulcer and stomach cancer are mainly caused by Helicobacter pylori. Thus, to diagnose the diseases, Helicobacter pylori test is exceptionally important.

Generally, Helicobacter pylori is tested by non-invasive means and invasive means. The non-invasive means includes C-13 Urea Breath Test, stool antigen test, and serological diagnosis. For a patient without symptom, these tests can be a preliminary test. Specifically, C-13 Urea Breath Test has a higher accuracy but cost much more. Stool antigen test and serological diagnosis have lower accuracies, but has a low cost to be suitable for normal health check-up.

In view of the invasive means, suitable for patients with symptoms, the doctor uses the gastroscopy to take the stomach tissue for rapid urease test, histological stain, or bacterial culture. These kinds of test can give more accurate result, but the medicine of the patient has to be stopped temporarily. Besides, the test costs much more and is time-consuming.

The present invention is, therefore, arisen to obviate or at least mitigate the above mentioned disadvantages.

### SUMMARY OF THE INVENTION

One of the objects of the present invention is to provide a test kit for human tissue which is easy to use and suitable for quick testing.

To achieve the above and other objects, the test kit for human tissue of the present invention includes a seat, a test paper, an adhesion layer, and a pressing piece.

The seat is formed with a receiving groove on a top face thereof. The seat is transparent at least at the receiving groove so that the receiving groove is visible from the outside. The test paper is disposed on the bottom of the receiving groove. The test paper is functional in testing and color indication so that a test result is able to be recognized by one outside. The adhesion layer has an adhesion face for adhering onto the top face of the seat to cover the receiving groove. The pressing piece has a first face and a second face opposite to each other. The first face is disposed on the adhesion face of the adhesion layer. The second face faces the seat and has a plurality of protruding portions. A position and a size of the pressing piece corresponding to a position and a size of the receiving groove. After placing a human tissue into the receiving groove and making the adhesion layer adhere onto the top face of the seat, the second face of the pressing piece presses the human tissue to squeeze fluid in the human tissue to make the test paper wet so as to undergo a specific test and to be recognizable from a bottom of the seat.

In some embodiments, an inner wall of the receiving groove is formed with a plurality of abutting pieces near the bottom of the receiving groove. Each of the abutting pieces radially extends toward the receiving groove from the inner wall of the receiving groove. The abutting pieces are spacedly arranged to be battlements-shaped. The test paper is arranged among the abutting pieces to be fixed by the abutting pieces. Preferably, a concave portion is formed between any two adjacent said abutting pieces.

In some embodiments, the seat is further formed with a relief channel. One end of the relief channel is opened at the inner wall of the receiving groove and extends to one of the concave portions. An other end of the relief channel communicates outside. The inner wall of the receiving groove is further formed with a gas channel extending from the opening of the receiving groove to an other said concave portion. A first chamfer portion is formed between the first face of the pressing piece and a peripheral wall of the pressing piece. A gas room is enclosed by the first chamfer portion and the adhesion layer. When the pressing piece is located in the receiving groove, the gas room communicates with the opening of the relief channel and the gas channel so that the gas compressed by the adhesion layer, the pressing piece, and the human tissue is able to be discharged via the gas channel, the gas room, and the relief channel.

In some embodiments, a second chamfer portion is formed between the inner wall of the receiving groove and the bottom of the receiving groove. The second chamfer portion is recessed to form an arc concave portion. The abutting pieces are formed on the second chamfer portion.

In some embodiments, the receiving groove is protruded from the bottom of the seat downward to be easy for observation.

In some embodiments, the protruding portions of the pressing piece are distributed evenly over the second face to form a raised grain shape.

In some embodiments, the adhesion layer is made by restickable adhesive.

In some embodiments, the diameter of the inner wall of the receiving groove is reduced from the opening thereof toward the bottom thereof so that the inner wall of the receiving groove is an inclined face expanding outward. The diameter of the peripheral face of the pressing piece is reduced from the first face toward the second face so that the peripheral face of the pressing piece is an inclined face tapering outward.

Thereby, the test kit of the present invention can acquire the tissue fluid quickly, and is able to test and color for observation.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

The present disclosure can be more fully understood by reading the following detailed description of the embodiment, with reference made to the accompanying drawings as follows:

FIG. 1 is a stereogram of the present invention;

FIG. 2 is a partial top view of the present invention;

FIG. 3 is an A-A cross-section of FIG. 2;

FIG. 4 is a partial stereogram of the present invention;

FIG. 5 is a stereogram showing a pressing member of the present invention;

FIG. 6 is a lateral view showing a pressing member of the present invention;

FIG. 7 is a bottom view showing a pressing member of the present invention;

FIG. 8 is breakdown profile of the present invention;

FIG. 9 is a profile of the present invention;

FIG. 10 is an illustration of the present invention during relief.

### DETAILED DESCRIPTION OF THE PREFERRED EMBODIMENTS

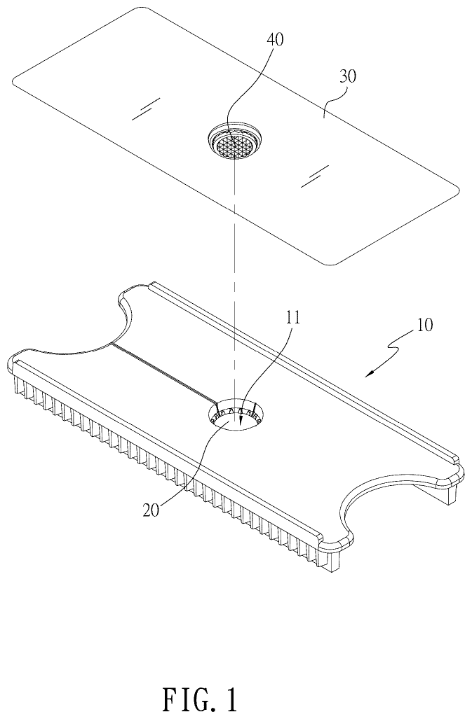

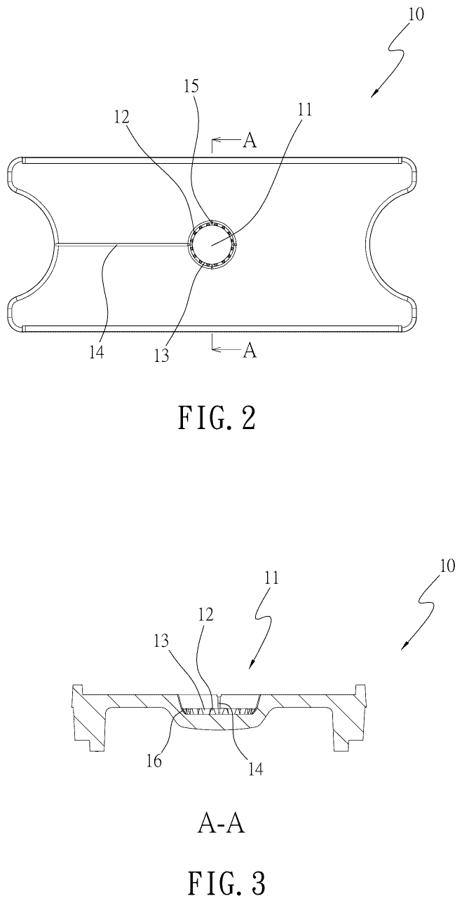

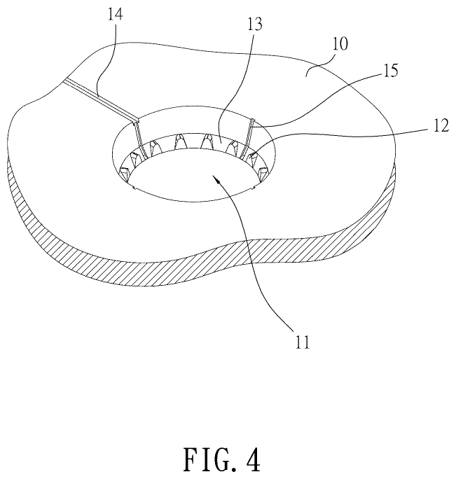

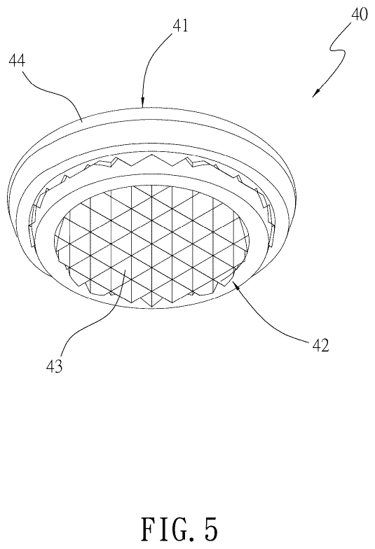

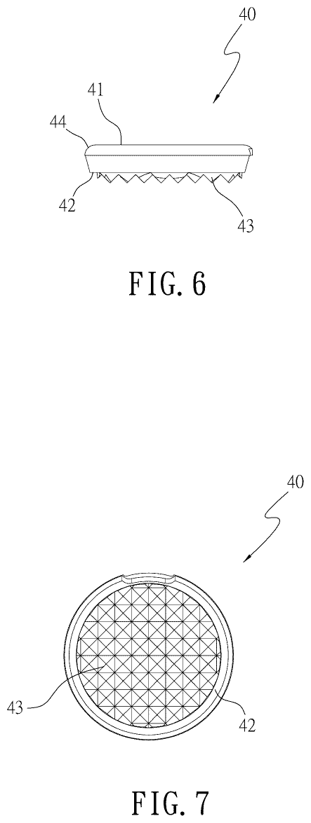

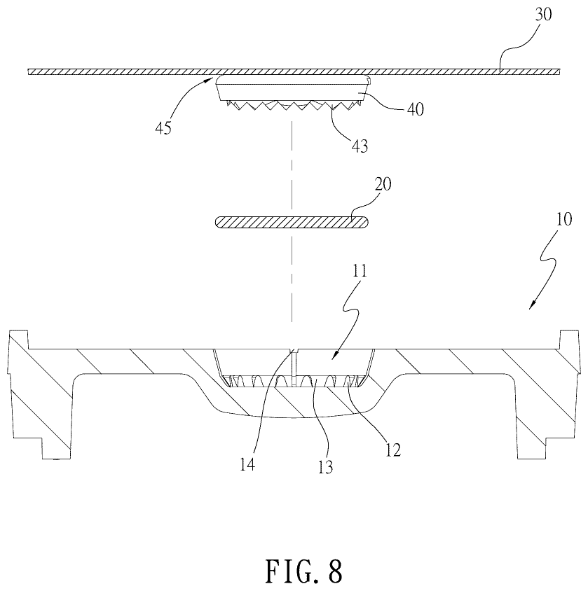

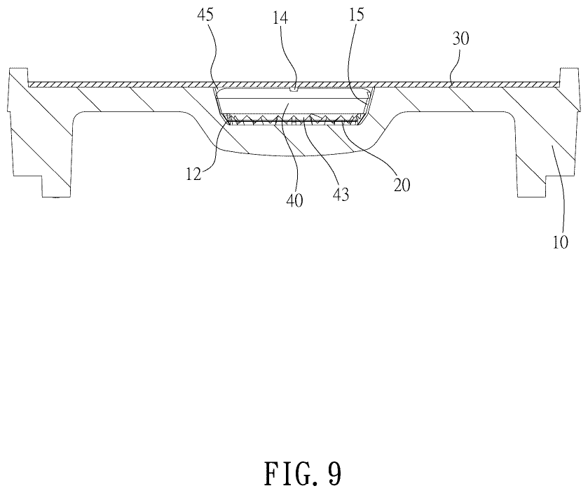

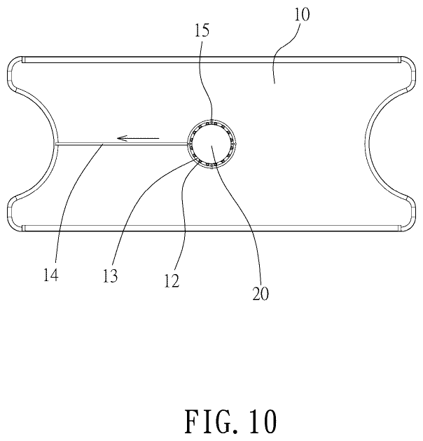

Please refer to FIG. 1 to FIG. 10, the present invention provides a test kit for human tissue, such as the stomach tissue, adapted for tests of specific chemical or biological characteristics. The test kit includes a seat 10, a test paper 20, an adhesion layer 30, and a pressing piece 40.

The seat 10 is formed with a receiving groove 11 on a top face thereof. The seat 10 is transparent at least at the receiving groove 11 to be visible from outside. Preferably, the seat is made of transparent material such as plastic or acrylic. In the present embodiment, the seat is wholly transparent, and the receiving groove 11 is protruded from the bottom of the seat 10 downward for easy observation.

The test paper 20 is disposed on the bottom of the receiving groove 11 and is functional in testing and color indication to notice the test result. For example, the test paper can be functional in testing Helicobacter pylori and color indication. In the present embodiment, an inner wall of the receiving groove 11 is formed with a plurality of abutting pieces 12 near the bottom of the receiving groove 11. Each of the abutting pieces 12 radially extends toward the receiving groove 11 from the inner wall of the receiving groove 11. The abutting pieces 12 are spacedly arranged to be battlements-shaped. The test paper 20 is arranged among the abutting pieces 12 to be fixed by the abutting pieces 12.

The adhesion layer 30 has a adhesion face to adhere onto the top face of the seat 10 to cover the receiving groove 11. For example, the adhesion layer 30 can be a plastic film or paper seat, and one face thereof is spread with adhesive to form the adhesion face. Preferably, the adhesion face is made of restickable material to be able to adhere onto the top face of the seat 10 repeatedly.

The pressing piece 40 has a first face 41 and a second face 42 opposite to each other. The first face 41 is disposed on the adhesion face of the adhesion layer 30. Preferably, the first face 41 is fixed onto the adhesion face of the adhesion layer 30. The second face 42 faces the seat 10 and has a plurality of protruding portions 43 thereon. Preferably, the protruding portions 43 of the pressing piece 40 are distributed evenly over the second face 42 to form a raised grain shape. The position and the size of the pressing piece 40 correspond to the position and the size of the receiving groove 11. After placing a human tissue into the receiving groove 11 and making the adhesion layer 30 adhere onto the top face of the seat 10, the second face 42 of the pressing piece 40 presses the human tissue to squeeze fluid in the human tissue to make the test paper 20 wet so as to undergo a specific test and to be recognizable from a bottom of the seat 10.

Practically, the adhesion layer 30 restickably adheres onto the top face of the seat 10, and the first face of the pressing piece 40 is fixed onto a position of the adhesion face corresponding to the receiving groove 11. In normal state, the adhesion layer 30 adheres onto the top face of the seat 10 to cover the receiving groove 11, and the pressing piece 40 is located in the receiving groove 11. When testing, the adhesion layer 30 is opened partially to reveal the receiving groove 11. At this time, the pressing piece 40 leaves the receiving groove 11. And then, the human tissue is placed into the receiving groove 11, and the adhesion layer 30 adheres onto the top face of the seat 10 again. At the same time, the pressing piece 40 enters the receiving groove 11 to compress the human tissue to squeeze the fluid to make the test paper 20 wet for further testing.

In the preferable embodiment, a relief structure is included. Practically, a concave portion 13 is formed between any two adjacent said abutting pieces 12. The seat 10 is further formed with a relief channel 14. One end of the relief channel 14 is opened at the inner wall of the receiving groove 11 and extends to one of the concave portions 13. The other end of the relief channel 14 communicates outside. The inner wall of the receiving groove 11 is further formed with a gas channel 15 extending from the opening of the receiving groove 11 to an other said concave portion 13. Preferably, plural gas channels 15 are included. A first chamfer portion 44 is formed between the first face 41 of the pressing piece 40 and a peripheral wall of the pressing piece 40. A gas room 44 is enclosed by the first chamfer portion 44 and the adhesion layer 30. When the pressing piece 40 is located in the receiving groove 11, the gas room 45 communicates with the opening of the relief channel 14 and the gas channel 15 so that the gas compressed by the adhesion layer 30, the pressing piece 40, and the human tissue is able to be discharged via the gas channel 15, the gas room 45, and the relief channel 14. Thus, the test result may not be interfered by the gas.

In detail, a second chamfer portion 16 is formed between the inner wall of the receiving groove 11 and the bottom of the receiving groove 11. The second chamfer portion 16 is recessed to form an arc concave portion. The abutting pieces 12 are formed on the second chamfer portion 16. Besides, the diameter of the inner wall of the receiving groove 11 is reduced from the opening thereof toward the bottom thereof so that the inner wall of the receiving groove 11 is an inclined face expanding outward. The diameter of the peripheral face of the pressing piece 40 is reduced from the first face 41 toward the second face 42 so that the peripheral face of the pressing piece 40 is an inclined face tapering outward.

In conclusion, the test kit of the present invention has a simple structure to be easy to manufacture. The test paper is difficult to fall out. The receiving groove is covered by the adhesion layer to prevent from contamination. In use, the adhesion layer is opened partially to reveal the receiving groove, and the human tissue can be placed into the receiving groove. After covering the receiving groove with the adhesion layer again, the pressing piece enters the receiving groove to squeeze the human tissue to make the test paper wet for testing. It is convenient and efficient. Besides, the relief structure can prevent the gas from interfering the test result.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A test kit for human tissue, including:
a seat, formed with a receiving groove on a top face thereof, the seat being transparent at least at the receiving groove so that the receiving groove is visible from the outside;
a test paper, disposed on a bottom of the receiving groove, the test paper being functional in testing and color indication so that a test result is able to be recognized by one outside;
an adhesion layer, having an adhesion face for adhering onto the top face of the seat to cover the receiving groove;
a pressing piece, having a first face and a second face opposite to each other, the first face being disposed on the adhesion face of the adhesion layer, the second face facing the seat and having a plurality of protruding portions, a position and a size of the pressing piece corresponding to a position and a size of the receiving groove; after placing a human tissue into the receiving groove and making the adhesion layer adhere onto the top face of the seat, the second face of the pressing piece presses the human tissue to squeeze fluid in the human tissue to make the test paper wet so as to undergo a specific test and to be recognizable from a bottom of the seat.

2. The test kit for human tissue of claim 1, wherein an inner wall of the receiving groove is formed with a plurality of abutting pieces near the bottom of the receiving groove, each of the abutting pieces radially extends toward the receiving groove from the inner wall of the receiving groove, the abutting pieces are spacedly arranged to be battlements-shaped, the test paper is arranged among the abutting pieces to be fixed by the abutting pieces.

3. The test kit for human tissue of claim 2, wherein a concave portion is formed between any two adjacent said abutting pieces.

4. The test kit for human tissue of claim 3, wherein the seat is further formed with a relief channel, one end of the relief channel is opened at the inner wall of the receiving groove and extends to one of the concave portions, an other end of the relief channel communicates outside, the inner wall of the receiving groove is further formed with a gas channel extending from the opening of the receiving groove to an other said concave portion, a first chamfer portion is formed between the first face of the pressing piece and a peripheral wall of the pressing piece, a gas room is enclosed by the first chamfer portion and the adhesion layer; when the pressing piece is located in the receiving groove, the gas room communicates with the opening of the relief channel and the gas channel so that the gas compressed by the adhesion layer, the pressing piece, and the human tissue is able to be discharged via the gas channel, the gas room, and the relief channel.

5. The test kit for human tissue of claim 2, wherein a second chamfer portion is formed between the inner wall of the receiving groove and the bottom of the receiving groove, the second chamfer portion is recessed to form an arc concave portion, the abutting pieces are formed on the second chamfer portion.

6. The test kit for human tissue of claim 1, wherein the receiving groove is protruded from the bottom of the seat downward to be easy for observation.

7. The test kit for human tissue of claim 1, wherein the protruding portions of the pressing piece are distributed evenly over the second face to form a raised grain shape.

8. The test kit for human tissue of claim 1, wherein the adhesion layer is made by restickable adhesive.

9. The test kit for human tissue of claim 1, wherein the diameter of the inner wall of the receiving groove is reduced from the opening thereof toward the bottom thereof so that the inner wall of the receiving groove is an inclined face expanding outward, the diameter of the peripheral face of the pressing piece is reduced from the first face toward the second face so that the peripheral face of the pressing piece is an inclined face tapering outward.
